(19) United States
(12) Patent Application Publication
(10) Pub. No.: US 2014/0127194 A1
(43) Pub. Date: May 8, 2014

(54) COMBINED PHARMACEUTICAL COMPOSITIONS FOR THE TREATMENT OF TUMORS

(71) Applicant: HYD Rákkutató és Gyógyszerfejlesztö Kft., Budapest (HU)

(72) Inventor: Gábor Somlyai, Budapest (HU)

(21) Appl. No.: 14/122,385

(22) PCT Filed: Jun. 1, 2012
(86) PCT No.: PCT/HU2012/000047

§ 371 Date: Jan. 10, 2014

Related U.S. Application Data
(60) Provisional application No. 61/497,534, filed on Jun. 16, 2011.

(30) Foreign Application Priority Data
Jun. 1, 2011 (HU) P1100290

(51) Int. Cl. A61K 33/00; A61K 31/282; A61K 31/7068; A61K 39/395; A61K 31/519; A61K 31/7048; A61K 31/4745; A61K 31/337; A61K 33/24

(52) U.S. Cl. CPC A61K 33/00; A61K 31/337; A61K 31/282; A61K 31/7068; A61K 33/24; A61K 31/519; A61K 31/7048; A61K 31/4745; A61K 39/39558
USPC 424/133.1; 424/600; 424/649

(57) ABSTRACT

The invention relates to the use deuterium depleted water (DDW) for the preparation of combined pharmaceutical compositions for the prevention or treatment of tumorous diseases, where the composition comprises DDW with deuterium content of 0.01 to 135 ppm and one or more antineoplastic agent(s), optionally together with one or more usual pharmaceutical auxiliary material(s). Further, the invention relates to pharmaceutical compositions for adjuvant treatment of tumorous diseases, where the composition comprises DDW with deuterium content of 0.01 to 135 ppm and one or more antineoplastic agent(s), optionally together with one or more usual pharmaceutical auxiliary material(s). The invention also relates to aqueous pharmaceutical compositions usually applied in curing where the aqueous component is DDW with deuterium content of 0.01 to 135 ppm.

### TECHNICAL FIELD OF THE INVENTION

The invention relates to combined compositions which increase the efficacy of drugs suitable for treatment of tumor patients and which contain deuterium-depleted water (DDW) together with a known antineoplastic agent. The invention also relates to water-based pharmaceutical compositions containing DDW.

### BACKGROUND OF THE INVENTION

Malignant tumors are the second largest cause of death in developed countries. After decades of increase, tumor mortality could be stabilized in these countries and a minimal decrease could be achieved with certain types of tumors; but the global WHO prognosis forecasts further significant increase of mortality and morbidity of malignancies. In every country, adequate care of tumor patients means an ever increasing burden for the health care system, and so for the economy. Thanks to the great development of molecular biology in the last two-three decades, the biochemical and genetic processes, essential in the regulation of cell division, could be revealed. As a result, several antineoplastic agents, coming from purposeful development, have been brought to the market. In these cases, molecules inhibiting the function of proteins central to tumorigenesis have been designed in knowledge of the 3-dimensional structure of these proteins; or other proteins, blocking the receptors involved in signal transmission, have been developed. (In earlier decades, the development of cancer treatment agents had been based on screening thousands of molecules and searching for the one which killed cancer cells. This partly explains the grave, occasionally life-threatening side effects of such medicines.) Having these important achievements of basic science and drug research in mind, it is even less understandable why the more and more exigent problem of cancer cannot be coped with. WHO forecasts 10 million deaths for the year 2020, in contrast to the 6 millions in 2010, although 200-300 new antitumor drugs are being tested in the clinical phase year by year.

The research which commenced in the early 90's marked a novel direction of antitumor drug development. The development is based on the recognition of the important role of naturally occurring deuterium (D) in the regulation of cell division. In the last year, patents based on Hungarian priority date have been granted in numerous countries (e.g. equivalents of the Hungarian patents HU208084 and HU 209787); and scientific papers were published (Gábor Somlyai, Gábor Jancsó, György Jákli, Kornélia Vass, Balázs Barna, Viktor Lakics and Tamás Gaál (1993) Naturally occurring deuterium is essential for the normal growth rate of cells. FEBS Lett. 317, 1-4.; Gábor Somlyai, G. Laskay, T. Berkényi, Gy. Jákli, G. Jancsó (1998) Naturally occurring deuterium may have a central role in cell signalling. Synthesis and Application of Isotopically Labelled Compounds 1997. Edited by J. R. Heys and D. G. Mellilo © 1998 John Wiley & Sons Ltd.; Gábor Somlyai (1998) Csökkentett deutérium-tartalmú viz, Új lehetöség a daganatterápiában. Komplementer Medicina II. évf. 6.szám, 6-9.; G. Somlyai, G. Laskay, T. Berkényi, Z. Galbács, G. Galbács, S. A. Kiss, Gy. Jákli, G. Jancsó (1998) The Biological Effects of Deuterium-Depleted Water, a Possible New Tool in Cancer Therapy. Z. Onkol./J. of Oncol. 30, 4; G. Somlyai, Z. Gyöngyi (2000) Deuterium Depletion can Decrease the Expression of c-myc, Ha-Ras and p53 Gene in Carcinogen-Treated Mice. In vivo 14:437-440; Berkényi T., Szabó M., Jákli Gy., Jancsó G., Somlyai G. (2000) A deutérium depléció elvének alkalmazása a daganatterápiában [The application of Dd-water in veterinary practice]. KisállatPraxis 2000/4, 1, 4:24-28; G. Somlyai, G. Jancsó, Gy. Jákli, T. Berkényi, Z. Gyöngyi, I. Ember (2001) The Biological Effect of Deuterium Depleted Water, a Possible New Tool in Cancer Therapy. Anticancer Research 21:1617; Szabó M., Sápi Z., Berkényi T., Somlyai G. (2003) A deutérium-megvonás hatása állati tumorokra és azok pathológiás képére [The Effect of Deuterium Depletion on Animal Tumors and their Pathological Pattern]. Az állatorvos III (7-8):22-23, 26-27.; Krisztina Krempels, Ildikó Somlyai and Gábor Somlyai (2008) A retrospective evaluation of the effects of deuterium depleted water consumption on four patients with brain metastases from lung cancer. Integrative Cancer Therapies 7(3):172-81; Somlyai, G. (2007) A hidrogén/deutérium izotóppár biológiai jelentösége—a deutériumdepléció daganatellenes hatása/Deuterium, as a key element in cell growth regulation. Biokémia, 31: 28-32; Györe I., Somlyai G. (2005) Csökkentett deutérium tartalmú ivóvíz hatása a teljesitökepességre sportolóknál/The effect of deuterium depleted drinking water on the performance of sportsmen. Sportorvosi Szemle/Hungarian Review of Sports Medicine 46/1:27-38; Somlyai, G. (2005) A deutérium depletio elvének alkalmazása a daganatterápiában és a megelözésben. Látlelet, XLIX(10):8; Somlyai, G. (2004) A deutérium depletio hatása IV. stádiumban lévö, emlötumoros betegek várható túlélésére/Deuterium Depletion and its Impact on Life Expectancy of Patients with Stage IV breast tumor. Komplementer Medicina/Journal of Complementary and Alternative Medicine VIII (4):30-35).

In oncological therapy, similarly to the treatment of other diseases, one way to increase efficacy is to combine two or three drugs to increase the chance of healing. In oncotherapy this procedure is especially indicated because a great deal of the cases is recognized too late to achieve complete healing with the means available at present. The chances of healing are further decreased by the heterogeneity of the tumors, and the often developing resistance. Heterogeneity can be present within the tumor because central and marginal cells can express markedly different genes, and is further increased by the genetic difference between the cells detaching from the primary tumor and forming metastases at distant locations of the organism, and the primary cells. This can cause that a given medicine is acceptably effective against the primary cancer but fully ineffective against the metastases. The drug sensitivity is changing even in the course of chemotherapy. Resistance may develop against one drug so that another one with different site of action needs to be given. In prostate cancer patients, for example, it is typically observed that antihormone chemotherapeutics are efficient in the first 1-2 years but later hormone resistant cancer cells develop on which the previous treatment has no effect.

These observations and the modest progress in oncology up to now point out that, on the one hand, new drugs need to be developed for maximal efficacy, and on the other hand efficacy should be increased by means of combining existing therapeutic means.

Median survival time (MST) is among the most important parameters when evaluating clinical trials. MST is the time span within which half of the patients die. By comparing survival data of groups receiving various treatments it is examined by how many weeks or months could MST be prolonged by a given treatment. Thanks to the patient follow-ups of the last decades, exact statistical data are available on the survivals for a given tumor type, stage and treatment. A survey of the last decades' results (Tusnády G, Gaudi I, Rejto L, Kásler M and Szentirmay Z: [Survival chances of Hungarian cancer patients in the National Cancer Registry] Magy. Onkol. 52: 339-349, 2008.) leads to the sad conclusion that the newer and newer drugs or combinations could increase MST in the majority of cases minimally, with a handful of weeks or 1-2 months.

Research with deuterium-depleted compositions (DDW) verified the antitumor effect of deuterium depletion, but the studies performed in vitro or in experimental animals, with DDW applied alone, gave no hint to effects in humans, nor to possible effects of combining DDW with routine antineoplastic drugs. The heterogeneity of antitumor drugs (alkalizing agents, antimetabolites, tyrosine kinase inhibitors, immunomodulators, hormonal effect compositions, angiogenesis inhibitors etc.), their dissimilar sites of action, and the frequently severe side effects further complicate the question. Experiments in vitro or in mice provide no reliable information on how the chance to survive is altered if the cytostatic drug (often causing severe side effects, destroying bone marrow function and the immune system) is combined with DDW.

We have no information on how the antitumor effect of DDW manifests in vivo in combination with cytostatics, and how the expectable life span is affected. This question is especially important for patients showing low white blood cell count due to cytostatic treatment. So the question is, whether co-administration of DDW can increase the efficacy of conventional treatments, and how DDW influences the efficacy of conventional drugs with partly unknown mechanisms of action.

In the last 18 years, nearly two thousand patients, receiving conventional antitumor treatment and consuming DDW, have been followed up. The cumulative length of patient follow-up, from diagnosis to end of following, is over five thousand years; and in a cumulative time span of more than two thousand years the patients consumed ca. 500 tons DDW. Two eminent conclusions of the recent experiences are the following: 1/ In the overwhelming majority of cases, DDW combined with conventional treatment significantly increased the patients' expectable life span; 2/ The efficacy of DDW could be maximized by minimizing intake of fluids with normal deuterium content. This fact is of great importance primarily because cancer patients may get several liters of infusion per day, partly for delivery of drugs and partly for protecting the kidneys, the liver etc. against harmful side effects of chemotherapy. Experience showed that omitting DDW even for one or two days in a treatment course, or substantially reducing its proportion within daily fluid intake, can significantly deteriorate its efficacy. EDDIG

### SHORT SUMMARY OF THE INVENTION

The invention relates to the use of deuterium-depleted water (DDW) for the preparation of combined pharmaceutical compositions for prevention or treatment of tumorous diseases, where the composition comprises DDW with deuterium (D) content of 0.01 to 135 ppm and one or more antitumor agent(s), optionally together with one or more usual pharmaceutical auxiliary material(s).

Formulation of the above composition can be one of the followings: infusion solution, injection, intraperitoneal solution, solution for irrigation, solution for inhalation or solution for enema.

The antitumor agent can be, advantageously, selected from among tyrosine kinase inhibitors, alkylating agents, antimetabolites, herbal alkaloids, cytotoxic antibiotics, monoclonal antibodies, antiandrogens, platinum compounds, topoismerase inhibitors, methyl hydrazides. More specifically, the agent can be Gleevec, 5-Fluorouracil, Taxotere, Taxol, Carboplatin, Herceptin, Tarceva, Gemzar, Cisplatin, Avastin, Alinta, Androcur, Estracyt, Zoladex, Xeloda, Doxorubicin, Irinotecan.

In one advantageous case, the combined composition comprises an antitumor drug for treatment and prevention of breast cancer, advantageously selected from among Taxotere, Taxol, Carboplatin, Herceptin.

In a further advantageous case, the combined composition comprises an antitumor drug for treatment and prevention of lung cancer, advantageously selected from among Tarceva, Gemzar, Taxol, Cisplatin, Alinta.

In a further advantageous case, the combined composition comprises an antitumor drug for treatment and prevention of prostate cancer, advantageously selected from among Androcur, Estracyt, Suprefact, Zoladex.

In a further advantageous case, the combined composition comprises an antitumor drug for treatment and prevention of pancreatic, colon and rectal cancer, advantageously selected from among Tarcev, Gemzar, Xeloda, Cisplatin.

Due to the interaction of DDW and the standard antitumor drugs, the latter can be applied in advantageous cases in doses reduced to 80-10% of the usual dose.

The invention also relates to combined pharmaceutical compositions for adjuvant treatment of tumorous diseases, which comprises DDW with deuterium (D) content of 0.01-135 ppm and one or more known antitumor agent(s), optionally together with one or more usual pharmaceutical auxiliary material(s).

In these compositions it is advantageous to use an antitumor agent mentioned at the above applications.

In advantageous cases, the composition contains the antitumor drug in reduced dose which is 80-10% of the standard dose.

Further, the invention relates to pharmaceutical compositions for adjuvant treatment of tumorous diseases, where the composition comprises DDW with deuterium (D) content of 0.01 to 135 ppm and one or more known antitumor agent(s), optionally together with one or more usual pharmaceutical auxiliary material(s).

The invention also relates to aqueous compositions usually applied in therapy where the aqueous component is DDW with deuterium (D) content of 0.01 to 135 ppm. Such composition is advantageously formulated as solution, cream or gel, e.g. as isotonic infusion stock solution.

The invention further relates to a method for prevention or treatment of tumorous diseases in which deuterium-depleted water (DDW) with 0.01-135 ppm deuterium (D) content is administered together with one or several antitumor agent(s). Advantageous cases, of such implementation (i.e. the advantageous active agents) are identical to those mentioned above.

### DETAILED DESCRIPTION OF THE INVENTION

The invention has been based on the striking discovery that by applying some conventional cancer therapies and deuterium depletion together survival time can be prolonged not only by a few weeks or 1-2 months but it can be multiplied. Another base of the invention is the knowledge that at certain types of tumors the combination of D-depletion and conventional therapy can result in two-three times longer MST compared to the survival of patients receiving conventional treatment only.

A further base of the invention is that in case of certain tumor types, regarded as incurable, the combined pharmaceutical composition (conventional treatment+deuterium depletion) can result in complete recovery. Still another base of the invention is the knowledge that integrating DDW into the system of oncological treatments can increase the percentage of completely healed patients, because the combined pharmaceutical composition given as follow-up treatment to patients in remission can reduce the frequency of relapsing patients to a fraction of the usual.

Still another base of the invention is the experience that the combined pharmaceutical composition could substantially improve the patients' life expectancies even if the conventional drugs could not be given in the desired protocol or could be given only in reduced doses or in reduced number of sessions due to their severe side effects. The substantially longer MST in spite of the reduced amount indicated that the combined pharmaceutical composition increases the efficacy of the antitumor drug to such extent that the concentration of these agents—being mostly toxic and causing severe, often lethal side effects—could be largely diminished in the combined composition while the effect is stronger than with the conventional preparation only. This experience allows the reduction of the active agents in the standard preparations used by now to 80-10% of the original, e.g. to 80, 70, 60, 50, 40, 30, 20 or 10% of that.

From the above follows such an embodiment of the invention in which the standard oncotherapeutical preparations are manufactured in a medium (solution) with reduced deuterium content, by using DDW as vehicle, because DDW can substantially improve the efficacy of the conventional drugs, and the dosing of DDW also can be optimized this way. If the infusions, medicines and other auxiliary compositions (infusions, cytostatics, injections etc.) are produced using DDW, the results achieved by now can be further improved.

Our experience showed that the expedient way of implementing the invention is to use a composition of 85-105 ppm D content right after diagnosis, in the first 1-3 months of therapy, and reduce the D content of the composition by 20-40 ppm monthly or bi-monthly until total recovery is achieved.

The invention enables the application of more efficient combination compositions than before by using conventional oncotherapeutical composition and DDW together.

The combined composition of conventional antitumor agents+DDW according to the invention increases the efficacy of the conventional drug and so improves the survival chance of the patients. The base of that is that the mechanism of action of certain antitumor agents and DDW are synergistic by attacking the same processes while other drugs become more efficient because these and DDW interfere with cellular metabolism at fully different sites. The outcome is that cancer cells will show minimal or no resistance to the joint effect of conventional agents and DDW. A further advantage of the new combined compositions described in the invention is that it can increase the sensitivity of therapy resistant tumors so that cancers refractory to standard treatment can also be eliminated. A further advantage is that the application of conventional agents made up in DDW enables the continuous lowering of the bodily D content resulting in another significant improvement of effect.

A further advantage of the new combined compositions described by the invention is that in using them for follow-up treatment of patients in remission the rate of relapse can be greatly reduced, and that can be achieved by compositions containing only 80-10% of the usual dose of the conventional agents, e.g. 80, 70, 60, 50, 40, 30, 20 or 10%, and so the side effects of the combined composition are greatly diminished in severity or are fully avoided.

The compositions according to the invention optionally may contain beyond the conventional agents and DDW also one or more inert, nontoxic auxiliary material(s) (e.g. vehicles, moisteners, sweeteners, aromas, buffers etc.). The composition can be formulated for oral (tablets, solution, emulsion, suspension etc.) or parenteral (e.g. infusion solution) application.

The compositions according to the invention can be produced by the known methods of pharmaceutical manufacturing, such as by mixing the agents and the organic or inorganic vehicle(s) and formulating the mixture into a galenical composition.

The daily dose of the pharmaceutical compositions described by the invention can be varied in a wide range, depending on several factors such as the patient's body weight and surface area, the kind of conventional agent, the deuterium level of DDW, the type of tumor and stage of disease etc.

The major advantages of the compositions and procedures described by the invention are as follows:

a/ The simultaneous effect of DDW and the antitumor composition prepared in DDW can increase the MST of the patients severalfold.

b/ In the follow-up treatment of patients in remission, the chance of relapse is reduced in the majority of cases by 50% at least.

c/ Preparing the standard antitumor drugs in DDW enables ongoing reduction of the D content of the patient's body during treatments, such as administering of infusions, which may substantially increase the efficacy of the treatment.

d/ Using DDW and conventional treatments together can diminish the side effects of the latter.

e/ In using DDW and conventional treatments together, 80-10% of the standard concentration of the conventional agent, such as 80, 70, 60, 50, 40, 30, 20 or 10%, can be applied at increased efficacy and greatly reduced toxicity.

f/ Conventional agents prepared with DDW may be of effect on tumors regarded earlier as refractory, and tumors diagnosed in late stage may become treatable.

g/ The application of conventional compositions made with DDW needs no special procedures.

h/ The compositions can be produced on industrial scale.

The invention also relates to water-based pharmaceutical compositions, characterized by using deuterium-depleted water (DDW) with 0.01-135 ppm D content as the aqueous constituent. Such a composition contains no antitumor agent (these are present in the implementation variants described above). These are kinds of pharmaceutical compositions by which water is delivered to the treated person's organism. The formulation can be solution, cream or gel, for example. Typical examples are infusions, like the isotonic infusion solutions. Such a water-based pharmaceutical composition may contain other active agents (which may be part of the patient's antitumor therapy but also may be totally independent of that).

The invention further comprises a method for prevention or curation of neoplasms where DDW of 0.01-135 ppm D content is given together with one or more antitumor drugs.

In this kind of implementation DDW and the agents are not necessarily administered on one composition. In one embodiment the DDW and the antitumor drug are used in two independent formulations. Of course, if the antitumor drug is given in an aqueous composition, it is practical and expedient to use DDW in this composition (the combined composition is detailed above), or to use the DDW-based aqueous composition also comprised in the invention.

In the above passage, “administering together” includes administration both simultaneously, at the same time, and in shifted times. The aim of such administration is to maintain the required concentration of the antitumor drug and reduced level of D in the body fluids, first of all in and around the tumor. It is of advantage if the patient requiring treatment consumes DDW beside the chemotherapy, and takes care of covering his/her entire water demand (including cooked food) by DDW.

The advantageous cases of this implementation (advantageous agents, eventual combined compositions and water-based compositions) are the same as described earlier.

### SUMMARY OF THE FIGURES

FIG. 1: Data of a 61 years old patient with prostate cancer and bone metastasis, with extremely high PSA level (1000 ng/mL). Beside conventional treatment, DDW was also applied. In spite of the potentially bad prognosis the patient remained in regression for 9 years. PSA level: left Y axis and broken line; DDW dose: right Y axis and solid line.

FIG. 2: 71 years old patient with prostate cancer and extreme PSA levels. The graph shows that periods of DDW consumption, that is, D depletion, always resulted in decreasing PSA level. Interrupting the DDW cure caused increasing PSA levels, and after finishing DDW consumption in December 2009 PSA was soaring. Broken line: PSA level, solid line: DDW dose.

FIG. 3: Survival curve of lung cancer patients with DDW treatment and with conventional therapy (solid line). The 129 cases receiving DDW plus conventional treatment had 22.5 months MST while those with conventional treatment only had 8 months MST. The two broken lines separately show the data of patients with the shortest and longest survival.

FIG. 4: Survival curve of stage IV breast cancer patients with DDW consumption and with conventional therapy (solid line). The 74 cases receiving DDW plus conventional treatment had 47.7 months MST while those with conventional treatment only had 20-22 months MST. The two broken lines separately show the data of patients with the shortest and longest survival.

FIG. 5: Curve 1 shows the growth of melanoma cells in medium with normal (150 ppm) D content. Curve 2 evidences that in medium with deuterium depleted water (85 ppm D) the growth of the cells was inhibited. Curves 3 and 5 show inhibitory effect of doxorubicin, 450 and 900 nM, in normal (150 ppm D) medium. Curves 4 and 6 show the inhibition by DDW plus the mentioned two concentrations of doxorubicin. The results demonstrate that the combination of DDW and doxorubicin had higher inhibitory effect than the two agents separately.

### EXAMPLES

The invention is presented in detail by the following examples without intention to limit the claimed scope by them.

### Treatment Examples

The advantages of applying DDW and conventional agents together are shown by the following examples.

### Example 1

### Determination of the Appropriate DDW Dose

The antitumor effect of DDW can be achieved only if patients consume it in sufficient doses. To make DDW consumption of different patients comparable, regarding body weight, water consumption habits, and the D level of the DDW used, “deuterium depletion unit (DdU)” has been defined. DDW treatment will produce higher effect in the patient's organism if his/her body weight is lower, the consumed amount of DDW is higher, and its D content is lower. Consequently, the dose is determined as follows:

$$Dose ( DdU ) = \frac{150 - D concentration of DDW}{body weight ( kg )} \times volume of DDW ( L / day )$$

Examples: Patient of 60 kg, consuming 1.6 L of 65 ppm DDW: DdU=2.25. Patient of 80 kg, consuming 1.3 L of 105 ppm DDW: DdU=0.73.

The correlation of DdU values and the best results of DDW consumption were analyzed in depth in stage IV breast cancer patients (with at least one verified distant metastasis).

The effect of a treatment is usually described in four categories: complete remission (CR), no tumor can be detected; partial remission (PR), tumor reduced by at least 30%; no change (NC), no significant change in the tumor size; progressing disease (PD), tumor significantly grown.

First the patients were divided in two groups (DdU≧1 and DdU<I) and it was found that at DdU≧1 70% of the patients were in CR and PR (30% were NC and PD); while at DdU<1 31% were CR and PR and 69% were NC and PD.

Then it was examined at which minimal DdU value is the proportion of CR and PR higher than NC and PD. It was found that at DdU=0.6 the difference between the two outcomes is still significant (p=0.046).

So the first observation in DDW dosing is that the patients have to take a DDW amount equivalent to DdU=0.6 but values at or above 1 are more beneficial.

### Example 2

### Timing of the Change of DdU

The last years' experiences doubtlessly showed that cancer tissue is able to adapt to decreased D level. This means that even if the starting DdU was correctly determined by the above method, after consuming that dose for 2-5 months the tumors were able to grow again after a few months delay. Hence, DdU has to be increased in the course of treatment, to make the D concentration in the body keep decreasing so that the tumor cells cannot adapt to the therapy. It is advantageous to increase DdU by 0.3-0.4 every 1-3 months, primarily by means of reducing the D content of the DDW by 15-20 ppm. This way of dosing can provide for continuously decreasing D level in the patient's organism or 8-10 months which can result in complete healing.

### Example 3

Prostate cancer is a frequent tumor type. Complete recovery has a chance only if operative removal of the organ is done in due time. Unfortunately, the disease of the majority of patients is diagnosed in a much later stage where an exclusively operative solution is impossible, and hormone, radiation or cytostatic therapy is done in the late stage. The disease reacts typically well on the treatment in the first 1-2 years but later hormone resistant cells appear and the drugs used by then lose effect. Distant metastases develop, first of all in bones, after which MST is merely 15-20 months.

The MST of 91 prostate cancer patients receiving DDW plus conventional treatment was found 11.02 years, although 46 of the 91 developed distant metastasis during the follow-up time. Analyzing further the data of homogenous subgroups of the patients, the following results were obtained.

a/ In 20 patients who developed bone metastasis within one year from diagnosis, MST was 64.8 months. In patients receiving conventional treatment only, MS was 15-20 months.

b/ In 12 patients who developed bone metastasis later than one year from diagnosis, MST could not be calculated because there were only 2 deaths in the 103 years of cumulative follow-up time.

c/ For the 45 cases without distant metastasis, MST could not be calculated either because there were “merely” 4 deaths in the 157 years of cumulative follow-up time.

These results demonstrate that the adequate combination of DDW and conventional therapy multiples the expectable survival of the patients. The recommended DdU values during the treatment were determined as described in Example 1 and 2.

Practical application of the method in Example 1 and 2, and the achievable results, will be shown by two prostate cancer cases in which the relationship of changing DdU and the levels of PSA (Prostate Specific Antigen) tumor marker prove the antitumor effect of consuming DDW.

The exceptional efficiency of combining DDW with conventional treatment is shown also by the fact that, at the time of submitting the patent claim, both cases were still alive, 9 and 6 years after being diagnosed, although MST in this stage is only 15-20 months.

A/ The first case shows the high efficacy of combining DDW and conventional therapy in prostate cancer diagnosed in late stage.

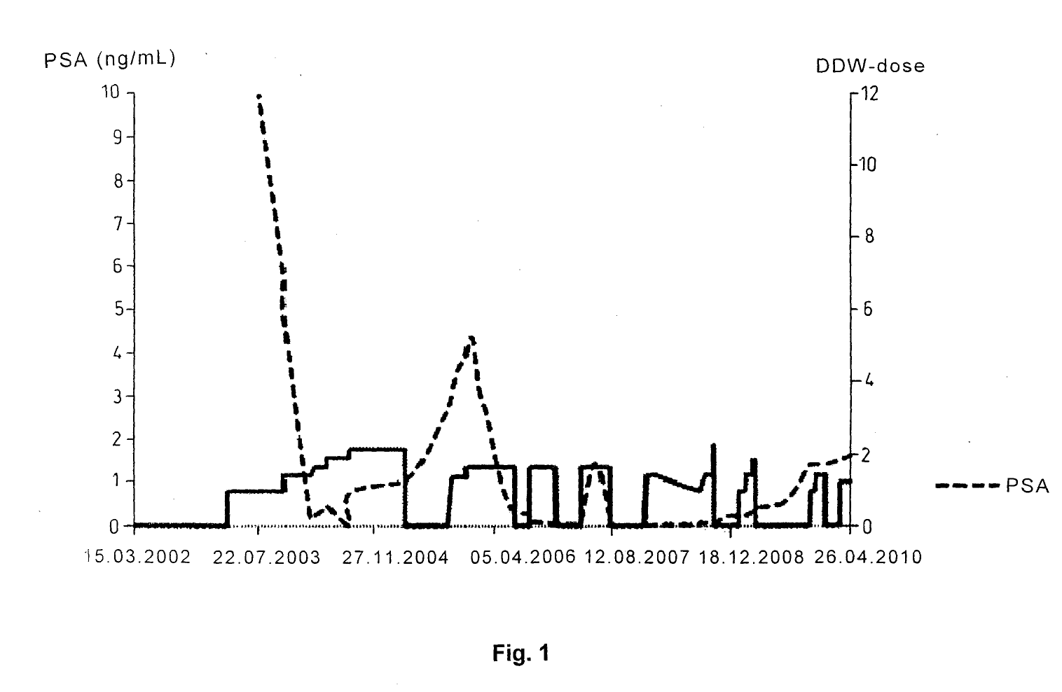

Patient 2 (61 years, FIG. 1) had bone metastasis in the early phase of the disease, PSA was over 1000 ng/mL. Due to the late stage, the patient got Estracyt and Androcure and PSA was rapidly lowered. He started consuming DDW in April 2003, 640 days (=21.3 months) after diagnosing bone metastasis with 15.0 ng/mL PSA. He consumed DDW for nearly one year at DdU of 1.0. A break of 6 months followed this cure, during which the disease progressed and PSA increased from 1 to 4.34 ng/mL. A new DDW cure brought regression and PSA below 1 ng/mL. 4 years after diagnosis, Androcure was replaced with Suprefact. Further DDW treatments (see FIG. 1 for DdU values) PSA was decreased in the following 3 years and the patient had no complaints in spite of the existing bone metastasis and the generally bad prognosis. Repeated bone scintigraphy in 2006 and 2009 showed no progress of disease. PSA increased moderately and slowly because at that time the patient consumed no DDW but the highest PSA value was only 0.56 ng/mL. The patient got the advice to drink DDW again in a regime that increased DDW dose every 4-6 weeks in order to prolong the period of remission. At the filing date of the present patent application the patient was still in remission, 2,722 days (5 years) after the first treatment.

B/ The second case also shows the increased efficacy of combined DDW plus conventional treatment compared to conventional-only. There was clear causal relationship between PSA level changes and DDW consumption, and cessation of DDW resulted in PSA increase.

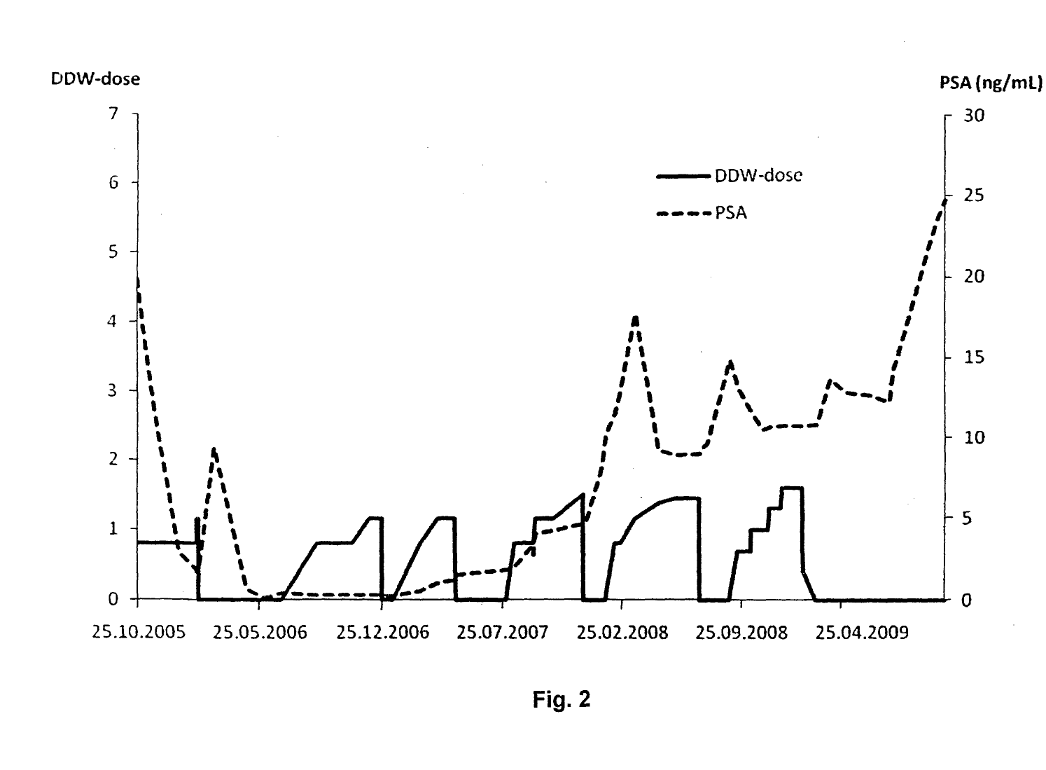

Patient 2 (71 years old) was diagnosed with prostate cancer and bone metastasis and had 580 ng/mL PSA in October 2005 (this earlier initial value is not shown in FIG. 2). The patient started DDW consumption only 7 days after diagnosis, so the first DDW cure and conventional treatment were simultaneous, and PSA decreased dramatically. PSA remained stable and low for one year but from April 2007 on it was over 1.0 ng/mL and kept increasing. But a massive PSA increase was seen only in December 2007 and July 2008 when the patient stopped DDW consumption. Due to increasing PSA, Androcure was changed to Suprefact in the conventional therapy. The subsequent DDW cures brought major PSA decrease twice (February to July 2008, September 2008 to January 2009) although later the level was steadily but moderately increasing. Finally, the disease progressed only slowly, but PSA increased sharply when the patient ceased to drink DDW in January 2009.

### Example 4

### Combination of DDW and Conventional Cancer Therapy in Lung Cancer Patients

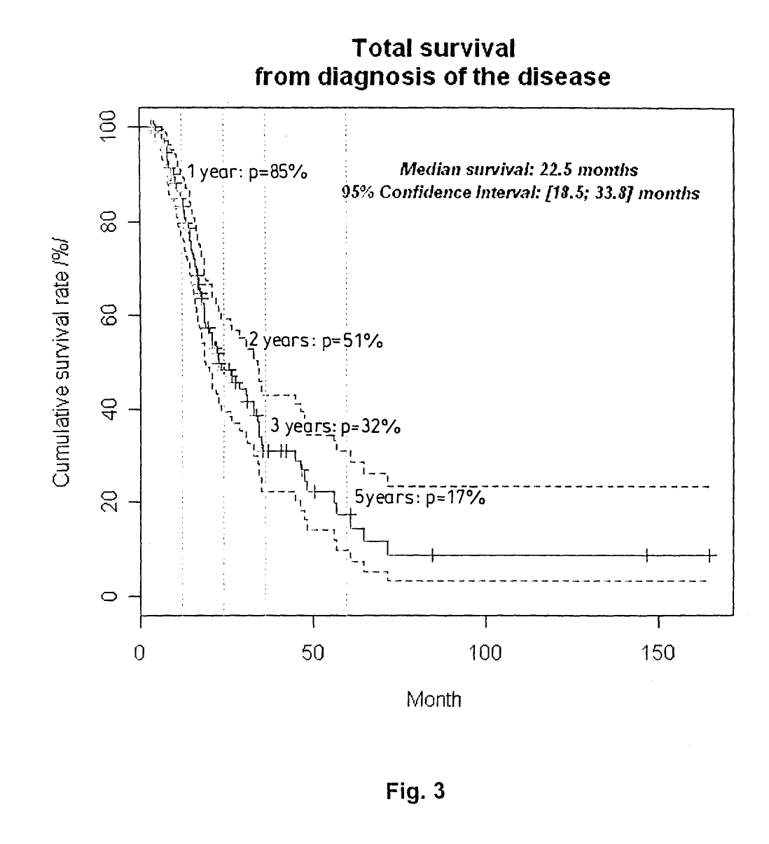

Lung cancer patients have MST of merely 8 months even with conventional therapy. Data of 129 lung cancer patients are analyzed here, of whom 72 were in stage IV because of distant metastases, and 57 were stage III. They received some conventional treatment and besides drank DDW for at least 90 days. These patients had 22.5 months MST despite the progressed stage (FIG. 3) because numerous patients died years (and not a few months) later or were completely healed. Beside DDW, the patients received chemotherapy according to their state, staging and tumor histology, in mono- or combined application, e.g. by Taxotere, Taxol, Carboplatin, Tarceva, Gemzar, Cisplatin, Avastin or Alinta. The nearly threefold increase in MST showed that the effect of conventional treatments was greatly increased by DDW.

DdU units used in the treatment were determined as described in Example 1 and 2.

Beside DDW, the patients received chemotherapy according to their state, staging and tumor histology, e.g. with the drugs and doses shown in Table 1.

TABLE 1

| Name of drug | Active agent | Dosing | |
|---|---|---|---|
| Taxotere | docetaxel | 60-100 | mg/m² |
| Taxol | paclitaxel | 135-175 | mg/m² |
| Paraplatin | carboplatin | 300-360 | mg/m² |
| Gemzar | gemcitabine | 1000-1250 | mg/m² |
| | hydrochloride | | |
| Platinol | cisplatin | 20-100 | mg/m² |
| Alimta | pemetrexed | 100-500 | mg/m² |
| VePeside | etoposide | 100-200 mg/m²/day for 1-5 days | |
| Xeloda | capecitabinum | 2500 mg/m² for 14 days, | |
| | | then 7 days break | |
| Irinotecan | camptosar | 60-100 mg/m² on day 1, 8 and 15 | |
| Adriamicyn | doxorubicin | 40-75 | mg/m² |
| | hydrochloride | | |

### Example 5

### Combination of DDW and Conventional Cancer Therapy in Lung Cancer Patients

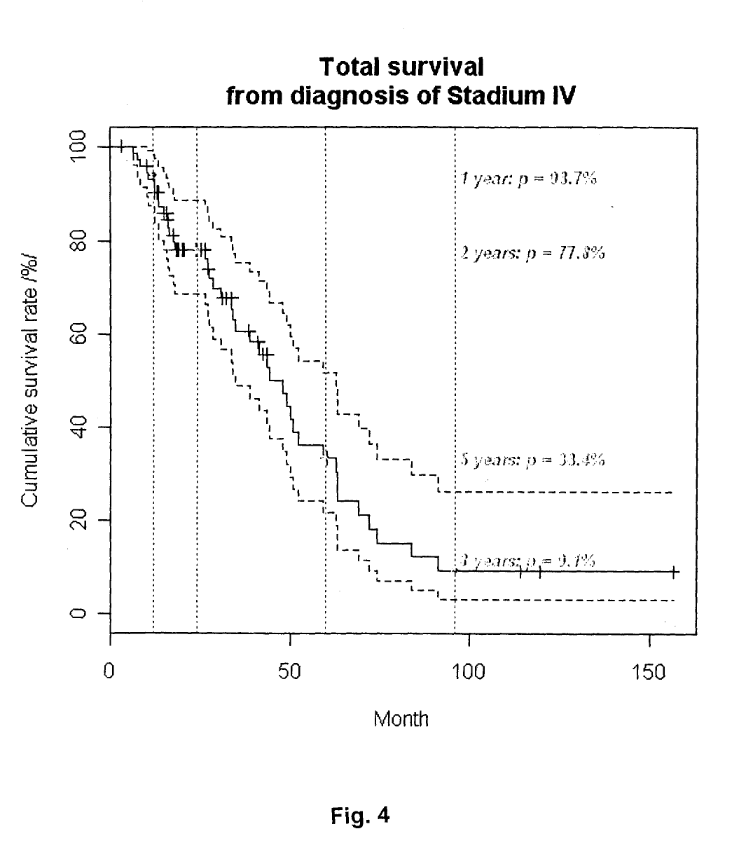

Data of stage IV breast cancer patients were analyzed. The 74 patients involved had at least on distant metastasis when DDW consumption started. The 74 patients had distant metastases in altogether 135 organs and the average time between diagnosis and start of drinking DDW was 4.8 years. Thus these patients were tumor-free for up to a few years but had already intensive conventional therapy at the start of DDW consumption because of the metastasis diagnosed ca. six months earlier. International literature suggested 20-22 months MST for this patient group, with 20% chance of 2 years survival. In our case, MST increased to 47.7 months and nearly 80% of the cases was still alive 2 years after the distant metastasis had been diagnosed (FIG. 4). With combined DDW plus conventional treatment, the outcomes and MST values among the patients were as follows:

| | |
|---|---|
| CR: 21.6% | MST: 63 months; |
| PR: 36.5% | MST: 50 months; |
| NC: 16.2% | MST: 35 months; |
| PD: 25.7% | MST: 31 months. |

The patients showing the rest reactions (CR) had three times longer MST, even those with progressing disease (PD) survived 50% longer.

Beside DDW, the patients received chemotherapy according to their state, staging and tumor histology, e.g. with the drugs and doses shown in Table 2.

TABLE 2

| | | |
|---|---|---|
| Name of drug | Active agent | Dosing |

| Taxotere | docetaxel | 60-100 | mg/m² |
|---|---|---|---|
| Taxol | paclitaxel | 135-175 | mg/m² |
| Carboplatin | carboplatin | 300-360 | mg/m² |
| Herceptin | trastuzumab | 2-8 | mg/kg |
| Gemzar | gemcitabine | 1000-1250 | mg/m² |
| Cisplatin | cisplatin | 20-100 | mg/m² |
| Alimta | pemetrexed | 100-500 | mg/m² |
| Zoladex | goserelin | 3.6 mg depo every 28 days | |
| Xeloda | Capecitabinum | 2500 mg/m² for 14 days, | |
| | | then 7 days break | |
| Doxorubicin | doxorubicin-HCl | 40-75 | mg/m² |

DdU units used in the treatment were determined as described in Example 1 and 2.

### Example 6

### Combined Use of DDW and Taxotere in the Treatment of Breast Cancer Patients

The homogenous subgroup of patients, receiving Taxotere therapy, was examined separately. The widespread application of Taxotere was crucial in raising with some months the previous shorter MST values to 20-22 months. Our experience was that MST of patients receiving Taxotere and consuming DDW increased to 35 months.

DdU units used in the treatment were determined as described in Example 1 and 2.

### Example 7

### Combination of DDW and Conventional Cancer Therapy can Reduce the Number of Relapsing Patients

Breast cancer is among those few tumors in which high rate of complete recovery is possible if the disease is diagnosed early, the tumor is surgically removed, and adequate follow-up treatment (chemo- and/or hormonal therapy) is applied to reduce the likelihood of distant metastasis. Unfortunately, the tumor is expected to reappear within 5 years in 50% of the cases despite all treatment. Among 48 such breast cancer patients who started to consume DDW within three months after successful surgery, the relapse rate diminished to 20% from the expected 50%. The results showed that if conventional follow-up treatment (chemo- radio- or hormone therapy) is combined with DDW, relapse rate can be substantially reduced. Those patients were relapsing, as shown by the data, who failed to consume DDW in recommended dose and time span. The follow-up treatment of these patients was also one of the above-mentioned therapies or the combination of them.

DdU units used in the treatment were determined as described in Example 1 and 2.

For patients in remission, DDW consumption besides standard conventional treatment can be as follows:

I/ DDW consumption together with conventional therapy, after successful surgical tumor removal, for 4-6 months;

II/ 2-3 months break in DDW consumption;

III/ A second DDW cure for 3-5 months, then 5-6 months break;

IV/ Another DDW cure for 2-4 months, repeated once a year for 4-5 years.

### Example 8

Among gastrointestinal tumors, pancreatic cancer shall be mentioned here, one of the most aggressive neoplasms. The disease is usually recognized in progressed stage, and the effect of the drugs can merely add a few months to the 4-6 months life expectancy of the patient. Five years survival is a few percent. The MST of the 36 pancreatic cancer patients in our data base was 19.6 months, although 20 of the 36 patients started DDW consumption in stage IV.

DdU units used in the treatment were determined as described in Example 1 and 2.

### Example 9

Besides the above analyses based on distinct tumor types, a few selected cases will be presented below where the combination of DDW and conventional treatment was exceptionally efficient.

a/ In certain breast cancer patients in whom genetic analysis showed hyperactivity of HER2 gene, the drug named Herceptin is applied. In one patient in our data base, a tumor of 7.5 cm maximal diameter was discovered in the mediastinum 10 years after mastectomy, in January 2002. The patient received Herceptin and started to drink DDW in February. 8 months later the tumor could not be detected and the person was free of symptoms 8 years later, in 2010.

b/ In a young female patient in 1998, chronic myeloid leukemia (CML) was diagnosed, and was medicated with Litaril because of high WBC count. The patient was consuming DDW from April 1999, her blood count improved steadily and the WBC count remained in the normal range from then on. In February 2001 the patient was enrolled in the clinical trial of the drug candidate STI 571 (Gleevec). By August 2001, bone marrow damage decreased to 66% from the previous 80%. In April 2002 the patient became fully negative and was free of complaints five years later.

c/ With another tyrosine kinase inhibiting agent, Sutent, a similar positive interaction was found. In one patient, kidney tumor was diagnosed in spring 2003 and was surgically removed. Up to spring 2006, three further operations were necessary because of the disease recidivating. Further tumors appeared later and were treated with Sutent which was efficient initially but in March 2007 MR images showed progression again. In autumn 2007 the patient started to consume DDW in parallel with Sutent, and the tumors shrank again. Images taken three years later showed ongoing decrease of the tumor sizes and the patient had no symptoms.

DdU units used in the treatment were determined as described in Example 1 and 2.

### Example 10

The above examples are supported by this in vitro result:

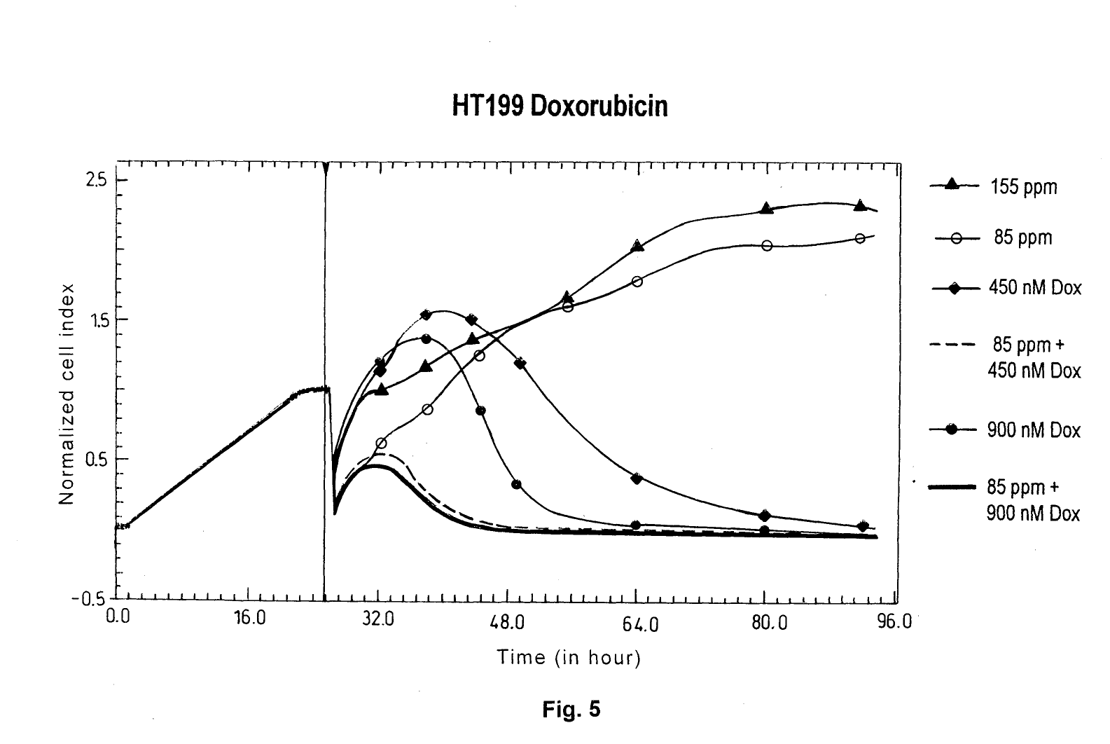

In the experiment, the effect of DDW, doxorubicin, and their combination was tested on HT199 melanoma cell line. The results are shown in FIG. 5.

Curve 1 shows the growth of melanoma cells in medium with normal (150 ppm) D content. Curve evidences that in medium with deuterium depleted water (85 ppm D) growth of the cells was inhibited. Curves 3 and 5 show the inhibitory effect of doxorubicin at 450 and 900 nM concentration in normal (150 ppm D) medium. Curves 4 and 6 show the inhibition by DDW plus the mentioned two concentrations of doxorubicin. The results demonstrate that the combination of DDW and doxorubicin had higher inhibitory effect than the two agents separately.

Examples for Compositions

Composition 1

Preparing Infusion Solution with DDW (for Agents in Dissolved Form)

The majority of antitumor compositions are administered to the patients in infusion form, which underlines the practical importance of this formulation.

In the production of the compositions according to the invention, DDW of 0.01-135 ppm D content (e.g. 105, 85, 65, 45, 35, 25, 5 ppm D) is used to prepare the infusion solution (the D level chosen according to the stage of disease and the time course of the treatment, see Examples 1 and 2).

The agent Docetaxel is used in 60-100 mg/m² dose, the concentrated agent is added to the DDW-based infusion as vehicle in dependence of the patient's body surface and the dose to be reached, and the final solution is the administered to the patient.

In case of Paclitaxel, the dose of 135-175 mg/m² can be administered identically.

With less stable antitumor agents, the infusion solution has to be prepared right before administration.

Composition 2

Preparing Infusion Solution with DDW (for Agents in Powder Form)

In the production of the compositions based on the invention, DDW of 0.01-135 ppm D content (e.g. 105, 85, 65, 45, 35, 25 or 5 ppm D) is used to prepare the infusion solution (D level chosen according to the stage of disease and the time course of the treatment, see Examples 1 and 2).

The applied dose of Carboplatin is 300-360 mg/m². For the patient, solution of 10 mg/mL is made using DDW, in a volume which provides the required dose according to the patient's body surface.

Trastuzumab is marketed in 150 mg units, from which infusion solution of 2-8 mg/kg body weight is made before administration, in DDW of D concentration corresponding to the treatment.

Composition 3

In the production of the compositions based on the invention, DDW of 0.01-135 ppm D content (e.g. 105, 85, 65, 45, 35, 25 or 5 ppm D) is used to prepare the usual pharmaceutical formulations but the dose of conventional drugs is lowered to 80-10% of the standard dose.

Composition 4

Physiological solution is prepared using DDW of 0.01-135 ppm D content (e.g. 105, 85, 65, 45, 35, 25 or 5 ppm D). To make the DDW-based saline, 9 g of NaCl is dissolved in 991 g of DDW with the chosen D concentration, and is processed to sterile infusion solution by the standard techniques of pharmaceutical industry.

Composition 5

The solutions for inhalation are produced by the usual techniques of this area, using DDW of 0.01-135 ppm D content (e.g. 105, 85, 65, 45, 35, 25 or 5 ppm D) as water component. The inhalation solution based on the invention can be applied together with the compositions used in the treatment of lung cancer patients.

Composition 6

Antitumor compositions show more main and less side effects if they are administered locally, directly into the organ suffering from malignancy. Such a procedure, TACE (trans-catheter arterial embolization) is used on liver metastases, developing especially frequently in colorectal cancer patients. In this case, the conventional agents are prepared as described at example compositions 1 and 2, and are administered to the affected organ after dissolution in DDW of 0.01-135 ppm D content (e.g. 105, 85, 65, 45, 35, 25 or 5 ppm D).

Composition 7

The isotonic solutions for intraperitoneal administration are prepared by standard methods, but the water component is DDW of 0.01-135 ppm D content (e.g. 105, 85, 65, 45, 35, 25 or 5 ppm D). The solution can be used with active agents as described at example compositions 1 and 2.

Composition 8

Solutions for irrigation are prepared by standard methods, but the water component is DDW of 0.01-135 ppm D content (e.g. 105, 85, 65, 45, 35, 25 or 5 ppm D). The irrigation solution based on the invention can be applied together with composition for treatment of gynaecological diseases.

1-5. (canceled)

6. Combined pharmaceutical composition for adjuvant treatment of tumorous diseases, which comprises deuterium-depleted water (DDW) with deuterium (D) content of 0.01 to 135 ppm and one or more antitumor agent(s), optionally together with one or more usual pharmaceutical auxiliary material(s).

7. The composition according to claim 6, where the antitumor agent is selected from the group consisting of tyrosine kinase inhibitors, alkylating agents, antimetabolites, herbal alkaloids, cytotoxic antibiotics, monoclonal antibodies, antiandrogens, platinum compounds, topoismerase inhibitors and methyl hydrazides.

8. The composition according to claim 6, where the antitumor agent is present in an amount reduced to 80-10% of the usual dose.

9. Aqueous composition usually applied in therapy, where the aqueous component is deuterium-depleted water (DDW) of 0.01-135 ppm deuterium (D) content.

10. Supplementary composition according to claim 9, which is formulated as solution, cream or gel.

11. Supplementary composition according to claim 10, which is an isotonic infusion solution.

12. Method for prevention or treatment of tumorous diseases, characterized by the fact that deuterium-depleted water (DDW) with 0.01-135 ppm deuterium (D) content is applied together with one or more antitumor agent(s).

13. The method according to claim 12, where the antitumor agent is selected from the group consisting of tyrosine kinase inhibitors, alkylating agents, antimetabolites, herbal alkaloids, cytotoxic antibiotics, monoclonal antibodies, antiandrogens, platinum compounds, topoismerase inhibitors and methyl hydrazides.

14. The method according to claim 12, where the antitumor agent is applied in an amount reduced to 80-10% of the usual dose.

15. The composition according to claim 7, where the antitumor agent is selected from the group consisting of Gleevec, 5-Fluorouracil, Taxotere, Taxol, Carboplatin, Herceptin, Tarceva, Gemzar, Cisplatin, Avastin, Alinta, Androcur, Estracyt, Zoladex, Xeloda, Doxorubicin and Irinotecan.

16. The composition according to claim 7, where the antitumor agent is present in an amount reduced to 80-10% of the usual dose.

17. The method according to claim 13, where the antitumor agent is selected from the group consisting of Gleevec, 5-Fluorouracil, Taxotere, Taxol, Carboplatin, Herceptin, Tarceva, Gemzar, Cisplatin, Avastin, Alinta, Androcur, Estracyt, Zoladex, Xeloda, Doxorubicin and Irinotecan.

18. The method according to claim 13, where the antitumor agent is present in an amount reduced to 80-10% of the usual dose.

19. The combined composition of claim 6 selected from infusion solution, injection, intraperitoneal solution, solution for irrigation, solution for inhalation or solution for enema.
